(19) United States
(12) Patent Application Publication
(10) Pub. No.: US 2026/0290593 A1
(43) Pub. Date: Sep. 24, 2026

(54) Immune Signature Detection Engine(s) For Predicting Neuroinflammatory Conditions Based On Antigen Receptor Sequences

(71) Applicant: The Regents of the University of Colorado, a body corporate, Denver, CO (US)

(72) Inventors: Graham Charles Peet, Denver, CO (US); Arjun Krishnan, Denver, CO (US); Gregory Parks Owens, Denver, CO (US); Jeffrey Lawrence Bennett, Denver, CO (US); Wendy Blair Macklin, Denver, CO (US)

(21) Appl. No.: 19/573,000

(22) Filed: Mar. 20, 2026

Related U.S. Application Data
(60) Provisional application No. 63/776,476, filed on Mar. 24, 2025.

(51) Int. Cl. G16H 50/20; G16B 30/00; G16B 40/20; G16H 15/00; G16H 50/70

(52) U.S. Cl. CPC G16H 50/20; G16B 30/00; G16B 40/20; G16H 15/00; G16H 50/70

(57) ABSTRACT

Systems and methods herein provide an immune signature (IS) detection engine and its related functions. In an example, an IS detection engine may receive antigen receptor sequences extracted from a patient sample, such as a blood sample from a patient. Responsive to receiving the antigen receptor sequences, the IS detection engine may process the antigen receptor sequences via one or more machine learning models, such as via an autoregressive language model. From the processing, the IS detection may identify an immune signature present within the antigen receptor sequences. Using the immune signature, the IS detection engine may detect a neuroinflammatory condition of the patient and/or generate a predicted diagnosis for the patient.

### CROSS-REFERNCE TO RELATED APPLICATIONS

This application claims priority to and the benefit of U.S. Provisional Application No. 63/776,476, titled IMMUNE SIGNATURE DETECTION ENGINE(S) FOR PREDICTING NEUROINFLAMMATORY CONDITIONS BASED ON ANTIGEN RECEPTOR, filed on March 24, 2025, which is hereby incorporated by reference in its entirety.

### TECHNICAL FIELD

Aspects of the disclosure are related to the field of computer software applications and services and, in particular, to an immune signature (IS) detection engine for detecting a neuroinflammatory condition within the antigen receptor sequences of a patient.

### BACKGROUND

Neuroinflammatory conditions encompass a diverse group of disorders characterized by immune-mediated inflammation of the central nervous system (CNS). These conditions, which include multiple sclerosis (MS), neuromyelitis optica spectrum disorder (NMOSD), and myelin oligodendrocyte glycoprotein antibody-associated disease (MOGAD), involve dysregulated immune responses that can lead to neuronal damage and functional impairment. While some disorders, such as NMOSD and MOGAD, are defined by specific autoantibodies, others, including MS, have more complex and heterogeneous immune signatures. MS, for example, is a chronic demyelinating disease with both T and B-cell involvement, but no single pathogenic antibody has been definitively linked to its onset or progression. The heterogeneity of immune mechanisms across neuroinflammatory diseases presents ongoing challenges in diagnosis, classification, and targeted therapy development.

Recent advances in immunological research have led to the identification of key cellular and molecular contributors to neuroinflammatory diseases, yet gaps remain in understanding their precise roles in disease pathogenesis. In MS, therapies targeting B-cells have demonstrated significant efficacy, highlighting their importance in driving inflammation and tissue damage. However, other neuroinflammatory conditions may be driven by distinct immune pathways, underscoring the need for disease-specific approaches. The complexity of these conditions is further compounded by genetic and environmental factors that shape immune responses over time. Despite substantial progress, the lack of a unifying biomarker or disease-defining immune signature for many neuroinflammatory diseases continues to hinder early diagnosis and the development of precision therapies.

Accordingly, there is a need for an immune signature (IS) detection engine, and its related functions, for predicting neuroinflammatory conditions based on a patient’s specific antigen receptor sequences present in the affected cells.

### SUMMARY

Systems and methods for providing an IS detection engine for detecting a neuroinflammatory condition. To detect a neuroinflammatory condition from a patient sample, which may be a blood sample, the IS detection engine may receive one or more antigen receptor sequences (a repertoire) prepared from the patient sample. Responsive to receiving the antigen receptor sequences, the IS detection engine processes the antigen receptor sequence(s) to identify an immune signature within or among the antigen receptor sequence(s). As described in greater detail below, the immune signature of the antigen receptor sequence(s) corresponds to a clonality or repertoire of a group of the antigen receptor sequences from the patient. Based on the identified immune signature, the IS detection engine generates a probability that the immune signature corresponds to one of a group of neuroinflammatory conditions. Using the probability, the IS detection engine may classify the antigen receptor sequences into a neuroinflammatory condition classification and generate a predicted diagnosis for the patient based on the neuroinflammatory condition classification.

This Summary is provided to introduce a selection of concepts in a simplified form that are further described below in the Technical Disclosure. It may be understood that this Overview is not intended to identify key features or essential features of the claimed subject matter, nor is it intended to be used to limit the scope of the claimed subject matter.

### BRIEF DESCRIPTION OF THE DRAWINGS

Many aspects of the disclosure may be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 illustrates an operational environment for providing an IS detection engine, according to an embodiment herein.

FIG. 2 illustrates an example architecture of an IS detection engine, according to an embodiment provided herein.

FIG. 3 illustrates a process for providing an IS detection engine and its related functions, according to an embodiment herein.

FIG. 4 illustrates an example operational environment of models leveraged by the IS detection engine to generate embeddings and determine neuroinflammatory conditions, according to an embodiment herein.

FIG. 5 illustrates an example IS training routine for training a language model used by the IS detection engine, according to an embodiment herein.

FIG. 6 illustrates an example architecture of an artificial intelligence (AI) model leveraged by the IS detection engine, according to an embodiment herein.

FIGS. 7A and 7B illustrate comparative evaluations of sequence embedding models to the AI model, according to an embodiment herein.

FIG. 8 shows an example computing apparatus suitable for providing an IS detection engine and related functions, according to an embodiment herein.

### DETAILED DESCRIPTION

Neuroinflammatory conditions represent a diverse group of disorders driven by immune-mediated inflammation within the CNS, often resulting in progressive neurological impairment, contributing to long-term disability and diminished quality of life. Neuroinflammatory conditions such as MS, NMOSD, and MOGAD collectively affect millions of individuals worldwide. While some neuroinflammatory conditions have well-characterized autoimmune targets, others remain poorly understood, lacking distinct immunological signatures or markers that define their underlying mechanisms. Beyond their direct neurological effects, neuroinflammatory conditions can significantly impact cognitive function and mental health, underscoring the need for improved diagnostic, classification, and treatment strategies. Despite advances in research and therapeutic development, many challenges persist in effectively managing these complex diseases.

MS serves as a prominent example of a chronic neuroinflammatory condition, affecting over two million people globally. As an inflammatory demyelinating disease, MS presents unique diagnostic and therapeutic challenges, despite its relatively high prevalence. The autoimmune component of MS is characterized by dysregulated immune responses involving B-cell receptors (BCRs) and immunoglobulins (Igs), which mistakenly target CNS tissues. Notably, therapies that deplete B-cells have demonstrated significant efficacy in reducing relapse rates, highlighting the critical role of this cell type in MS pathogenesis. However, despite extensive research, no definitive immune signature or disease-specific epitope has been identified, leaving key aspects of MS pathophysiology unresolved.

As such, diagnosis of MS remains complex, time-consuming, and costly, often requiring a combination of clinical evaluation, imaging, and laboratory testing. For example, patients typically undergo multiple MRI scans, lumbar punctures to assess cerebrospinal fluid abnormalities, and referrals to specialists for comprehensive assessment. Despite these measures, MS can be challenging to differentiate from other neurological conditions, leading to significant rates of misdiagnosis. Studies indicate that misdiagnosis occurs in approximately 20% of cases outside of specialized tertiary care centers, highlighting the limitations of current diagnostic criteria and methodologies. As a result, some patients may be subjected to unnecessary disease-modifying therapies, which can be costly and carry potential risks, while others may experience delays in receiving appropriate treatment for their actual condition.

Similar to MS, other neuroinflammatory conditions present significant challenges in diagnosis and treatment due to difficulties in identifying immune signatures within antigen receptors. Neuroinflammatory conditions such as NMOSD and MOGAD share overlapping clinical features with MS (e.g., similar symptoms and disease manifestations), often leading to delays or errors in diagnosis. While NMOSD is commonly associated with aquaporin-4 (AQP4) autoantibodies and MOGAD with myelin oligodendrocyte glycoprotein (MOG) antibodies, a subset of patients lack these immune signature or markers, complicating definitive diagnosis. Additionally, variations in immune responses across individuals contribute to heterogeneity in disease presentation, further obscuring clear immunological distinctions.

Although the discussion herein primarily focuses on MS as a representative example, it is important to recognize that the principles, techniques, and systems described herein apply broadly to other neuroinflammatory conditions that involve the adaptive immune system. The underlying issues related to immune receptor profiling, antigen specificity, and diagnostic uncertainty noted in the following disclosure extend beyond MS and are relevant to the broader spectrum of CNS inflammatory disorders, herein referred to as neuroinflammatory conditions generally.

To address at least the above shortcomings of conventional approaches to detecting and diagnosing neuroinflammatory condition, an example immune signature (IS) detection engine is provided herein. As will be expanded on in greater detail below, the IS detection engine processes blood samples of patients to identify immune signatures indicative of a particular neuroinflammatory condition. That is, based on the antigen receptor sequences, such as adaptive immune receptor sequences, present in the blood samples, the IS detection engine identifies an immune signature corresponding to a neuroinflammatory condition. In some embodiments, the IS detection engine is able to determine a patient’s disease status based on the immune signature, such as the patient’s antigen receptor sequence repertoire, present in the blood sample. By determining the patient’s disease status, the IS detection engine provides detailed information on a respective neuroinflammatory condition’s progression, allowing for tailored and targeted treatment plans, and avoiding unnecessary invasive tests and procedures.

The IS detection engine provides significant clinical and healthcare benefits over conventional diagnostic approaches to neuroinflammatory conditions. By identifying disease-specific immune markers, the IS detection engine facilitates earlier and more precise diagnoses, reducing reliance on expensive, invasive, and time-consuming procedures like lumbar punctures and repeated MRI scans. The improved diagnostic accuracy of the IS detection engine also minimizes misdiagnoses, ensuring that patients receive the appropriate treatments while avoiding unnecessary exposure to ineffective or harmful therapies. Additionally, the IS detection engine enhances disease monitoring, allowing clinicians to assess treatment response and disease progression with greater precision. Beyond MS, the IS detection engine is able to detect immune signatures of other neuroinflammatory conditions, helping to differentiate disorders with overlapping clinical presentations and refining personalized treatment strategies. Ultimately, the IS detection engine not only improves patient outcomes but also alleviates the economic burden on healthcare systems by streamlining diagnostic workflows and optimizing therapeutic interventions for a range of neuroinflammatory conditions.

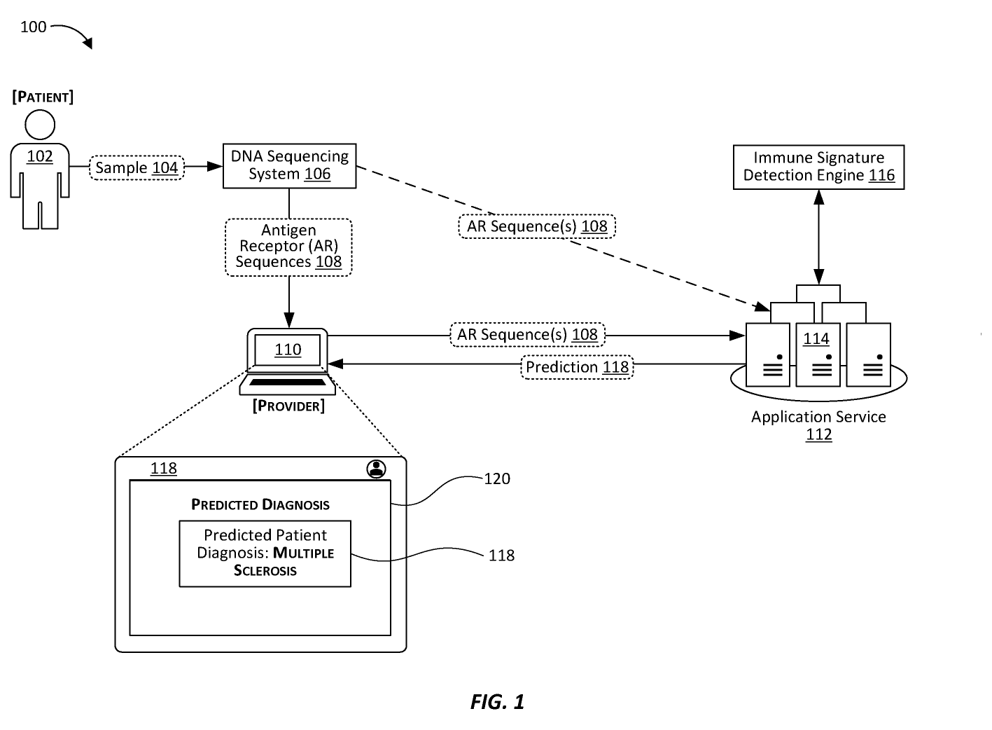

Turning now to FIG. 1, FIG. 1 illustrates an operational environment 100 for providing an IS detection engine, according to an embodiment herein. In particular, the environment 100 illustrates the IS detection engine 116 leveraged by a provider to augment diagnosis of a neuroinflammatory condition for a patient 102. As noted above, for ease of explanation, the following discussion focuses on MS as a specific example of a neuroinflammatory condition. However, it should be understood that this discussion is equally applicable to other neuroinflammatory conditions and conditions provoking an immune response, including acute Epstein-Barr virus (EBV) infection, hepatitis C virus (HCV) infection, SARS-CoV-2 infection, NMOSD, and MOGAD, as well as autoimmune disorders such as systemic lupus erythematosus (SLE). Additionally, this discussion is relevant to myasthenia gravis (MG), including anti-MuSK and anti-mAChR subtypes, respiratory inflammatory conditions such as asthma, and human immunodeficiency virus (HIV) infection.

To aid in diagnosing the patient 102 and/or developing a treatment plan to address the patient’s 102 symptoms or current disease status, a provider, via the client device 110, may use the IS detection engine 116. That is, in some embodiments, the IS detection engine 116 may be leveraged to detect a neuroinflammatory condition of the patient 102, while in other embodiments, the IS detection engine 116 may be leveraged to determine the progression or disease status of a neuroinflammatory condition. As used herein, a disease status refers to the current state or stage of a neuroinflammatory condition, including but not limited to its onset, progression, stabilization, remission, or relapse. By determining the disease status of a respective neuroinflammatory condition, the IS detection engine 116 can guide diagnosis, prognosis, and treatment decisions by evaluating the severity, activity, or expected trajectory of the condition. In some aspects of the present disclosure, the disease status is also referred to as a healthy status, reflecting the degree to which the patient’s 102 condition approaches a normal, non-diseased state.

To detect a neuroinflammatory condition or assess the disease status of an existing neuroinflammatory condition, a sample 104 may be collected from the patient 102. The sample 104 may be a blood sample or a cerebrospinal fluid (CSF) sample. Under conventional approaches, many neuroinflammatory conditions require CSF analysis for accurate detection and diagnosis. For example, in the context of MS, a CSF sample is required because MS cannot be reliably detected through a blood sample alone. However, obtaining a CSF sample is significantly more invasive and painful for patients compared to a routine blood draw. CSF collection requires a lumbar puncture (spinal tap), in which a needle is inserted into the lower spine to extract fluid, often causing discomfort, headaches, and potential complications. In contrast, a blood sample can be easily obtained through a simple venipuncture, making it a far less burdensome procedure for patients.

An advantage of the IS detection engine 116 is its ability to detect neuroinflammatory conditions using a blood sample. As will be described in greater detail below, the IS detection engine 116 is able to detect a neuroinflammatory condition using a blood sample, thereby providing a less invasive and less painful diagnosis process for patients, in particular when the neuroinflammatory condition is MS. While the following focuses on the sample 104 being a blood sample, in some embodiments, the sample 104 may be CSF, depending on the neuroinflammatory condition and disease status of the patient 102.

Once the sample 104 is acquired from the patient 102, the sample 104 may be submitted to a DNA sequencing system 106. It should be appreciated that while the DNA sequencing system 106 is illustrated as separate from the IS detection engine 116, in some embodiments, one or more components of the DNA sequencing system 106 may be part of the IS detection engine 116. The illustrated example, however, depicts an embodiment where the sample 104 is submitted to a third-party DNA sequencing system 106 to extract antigen receptor sequences 108 from the sample 104.

The antigen receptor sequences 108, which correspond to sequences encoding adaptive immune receptors, encompass a diverse set of B-cell and T-cell antigen receptors that play a central role in adaptive immunity. Depending on the various application, the antigen receptor sequences 108 may include a nucleotide or amino acid sequence corresponding to a B-cell receptor (BCR) immunoglobulin heavy chain (IGH) DNA sequence, which encodes the variable and constant regions of the BCR heavy chain, essential for antigen recognition and immune signaling. In another embodiment, the antigen receptor sequence(s) 108 may include an immunoglobulin light chain DNA sequence, selected from either the lambda (IGL) or kappa (IGK) chains (collectively referred to herein as IGL/K sequences), which pair with the heavy chain to form the complete antigen-binding structure of a functional BCR.

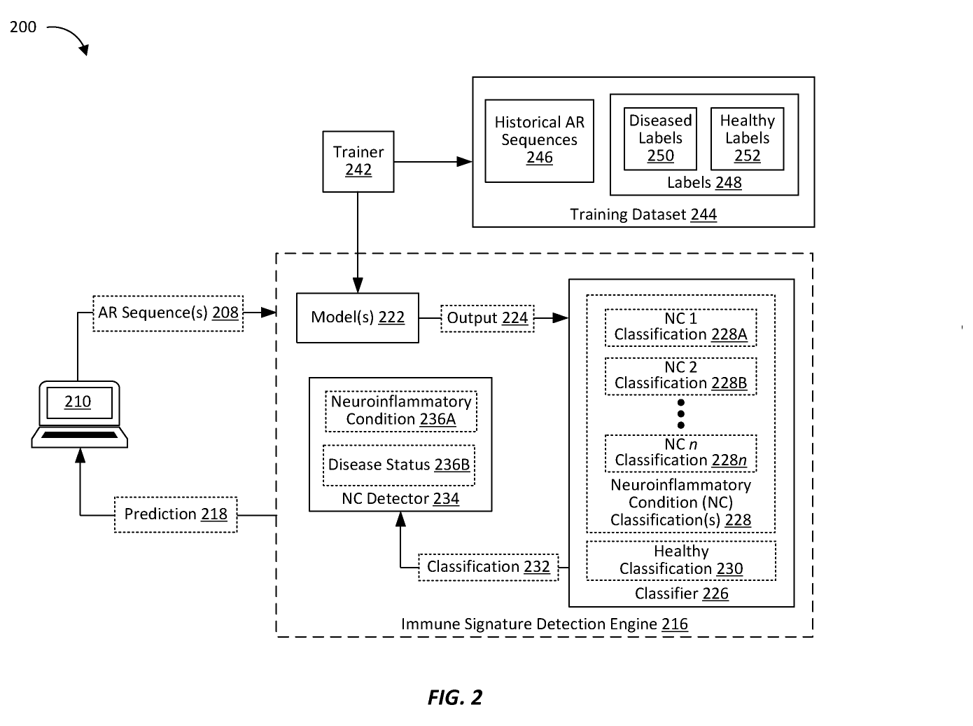

In yet another embodiment, the antigen receptor sequence(s) 108 may include a T-cell receptor (TCR) DNA sequence, selected from the alpha (TCRα) or beta (TCRβ) chains (collectively referred to herein as TCRα/β sequences), which form heterodimeric receptors on T-cells responsible for recognizing peptide antigens presented by major histocompatibility complex (MHC) molecules. As will be expanded on in greater detail below with respect to FIGS. 2-4, the unique recombination of the antigen receptor sequences within a respective patient’s 102 blood sample 104 generates an immune signature indicative of a neuroinflammatory condition.

To obtain antigen receptor sequences 108 from the sample 104, which in this example is a blood sample, the DNA sequencing system 106 may first isolate total RNA or messenger RNA (mRNA) from immune cells present in the sample 104. The DNA sequencing system 106 then generates complementary DNA (cDNA) through reverse transcription, using primers specific to conserved regions of immunoglobulin (Ig) or TCR genes. Next, targeted amplification of the antigen receptor sequences 108 is performed using multiplex PCR or RNA capture-based methods to enrich for variable regions of the BCR or TCR. The amplified antigen receptor sequences are then subjected to high-throughput sequencing, such as next-generation sequencing (NGS), to generate a comprehensive profile of antigen receptor diversity. By leveraging an antigen receptor sequence library, the DNA sequencing system 106 can map sequencing reads to known IGH, light chain (IGK/IGL), or TCR (TCRα/TCRβ) gene segments or sequences, enabling precise identification of the respective antigen receptor sequences 108 within the sample 104.

Once the antigen receptor sequences 108 are extracted from the sample 104, the DNA sequencing system 106 provides the antigen receptor sequences 108 to the provider for analysis. As illustrated, the client device 110 may be in operational communication with the application service 112 for one or more functions or features. Broadly speaking, the application service 112 provides software application services to end points, such as the client device 110, examples of which include medical software for detecting neuroinflammatory conditions or determining a disease status of patients, such as the patient 102. In some embodiments, the DNA sequencing system 106 may provide the antigen receptor sequence(s) 108 to the application service 112 or the IS detection engine 116 directly, depending on the application.

In the illustrated example, the application service 112 may provide a diagnostic tool for predicting a neuroinflammatory condition diagnosis, such as predicted diagnosis 118 for the patient 102. As such, the client device 110 may load and execute software applications locally that interface with services and resources provided by the application service 112, such as the IS detection engine 116. The applications may be natively installed and executed applications, web-based applications that execute in the context of a local browser application, mobile applications, streaming applications, or any other suitable type of application. Example services and resources provided by the application service 112 include front-end servers, application servers, content storage services, authorization and authentication services, and the like.

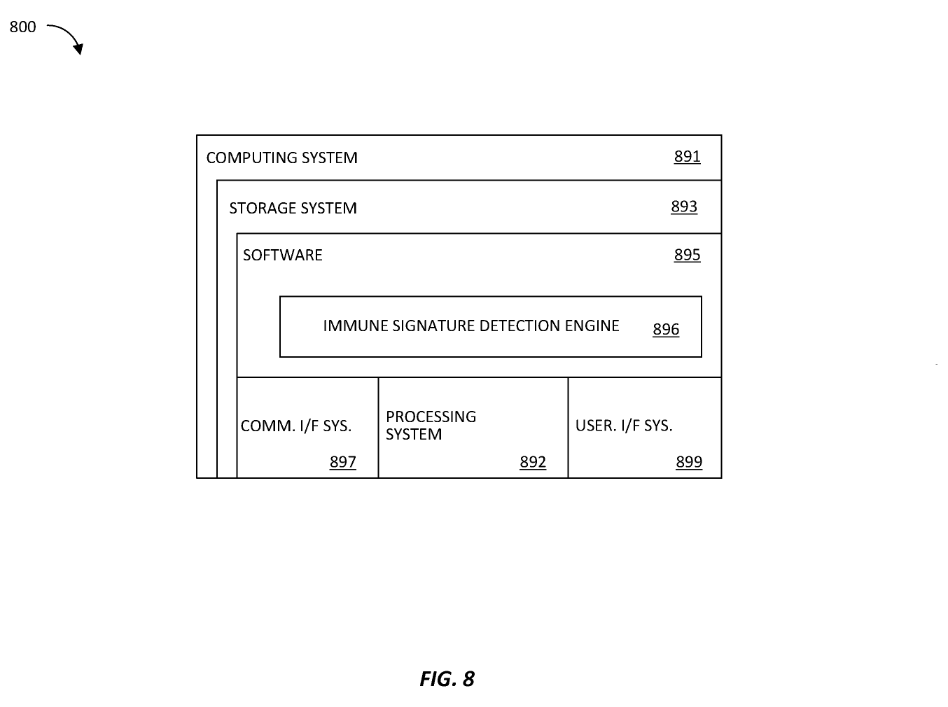

To interact with the application service 112, the client device 110 may communicate with the application service 112 via one or more internets and intranets, the Internet, wired and wireless networks, local area networks (LANs), wide area networks (WANs), or any other type of network or combination thereof. Examples of the client device 110 may include personal computers, tablet computers, mobile phones, gaming consoles, wearable devices, Internet of Things (IoT) devices, and any other suitable devices, of which computing apparatus 891 in FIG. 8 is also broadly representative.

In the illustrated example, the application service 112 operates in a cloud-based environment. As such, the application service 112 employs one or more server computers 114 co-located with respect to each other or distributed across one or more data centers to deliver its functionalities and services. Example servers include web servers, application servers, virtual or physical servers, or any combination or variation thereof, of which computing apparatus 891 in FIG. 8 is broadly representative.

As illustrated, the application service 112 may include an integration with the IS detection engine 116 to generate a prediction of a likely neuroinflammatory condition diagnosis for the patient 102, as described herein. In some embodiments, the IS detection engine 116 may be executed remotely by the application service 112 or a third party, while in other embodiments the IS detection engine 116 may be installed and executed locally on the client device 110. In still other embodiments, one or more functions of the IS detection engine 116, as described herein, may be installed and executed locally on the client device 110, while the remaining functions are integrated and executed remotely via the application service 112 or a third party.

As noted above, to detect a neuroinflammatory condition of the patient 102, the provider, via the client device 110 may submit the antigen receptor sequence(s) 108 to the IS detection engine 116, such as via the application service 112. Responsive to receiving the antigen receptor sequence(s) 108, the IS detection engine 116 may process the antigen receptor sequence(s) 108 to determine whether an immune signature indicative of a neuroinflammatory condition is present.

An immune signature may be or include one or more identifiable patterns, features, or relationships present within antigen receptor sequences or representations derived from antigen receptor sequences that are indicative of an immune response associated with a particular biological condition. The immune signature may be determined from individual antigen receptor sequences or from collective properties of a repertoire of antigen receptor sequences obtained from a biological sample of a patient.

In some embodiments, an immune signature may include sequence-level features, such as nucleotide or amino acid motifs, clonotypes, public or private clonality patterns, gene segment usage (e.g., V, D, or J segment usage), complementary-determining region (CDR) characteristics, or other structural or statistical properties of antigen receptor sequences. In other embodiments, the immune signature may be represented as one or more numerical representations derived from antigen receptor sequences, including sequence embeddings, repertoire-level embeddings, probability distributions, vector representations, or other learned feature representations generated by a machine learning model.

The immune signature may reflect relationships among antigen receptor sequences within a repertoire, including similarity relationships, clustering patterns, density distributions in an embedding space, or other relationships indicative of immune receptor diversity or clonal expansion. In certain implementations, the immune signature corresponds to features learned by a language model or other machine learning model that distinguish antigen receptor repertoires associated with particular disease states, immune responses, or biological conditions from repertoires associated with other conditions or healthy controls. An antigen receptor repertoire may be or include a collection of antigen receptor sequences present within a biological sample obtained from a patient. In some cases, the antigen receptor repertoire may represent the diversity and composition of B-cell or T-cell receptors present within the sample and may include thousands or more unique receptor sequences generated through immune recombination processes.

When an immune signature is present within the antigen receptor sequence(s) 108, the IS detection engine 116 may generate the predicted diagnosis 118 and provide the predicted diagnosis 118 to the client device 110. The predicted diagnosis 118 may indicate a likelihood that the patient 102 has a respective neuroinflammatory condition, such as MS. The provider may leverage the predicted diagnosis 118 to augment his or her evaluation of the patient’s 102 disease diagnosis or disease status. Detection of a neuroinflammatory condition, and responsive generation of the predicted diagnosis 118 by the IS detection engine 116 is described in greater detail below with respect to FIGS. 2-4.

As illustrated, the predicted diagnosis 118 may be displayed to the provider via a user interface 120 of an application executing on the client device 110. The application may correspond to the application service 112 or with an application associated with the IS detection engine 116. As will be described in greater detail below, the predicted diagnosis 118 may include a detected neuroinflammatory condition or detected disease status of the patient 102. Through the user interface 120, the provider can interact with the predicted diagnosis 118 to determine a final diagnosis for the patient 102 and/or develop a treatment plan or management strategy that optimally addresses the patient’s 102 current prognosis.

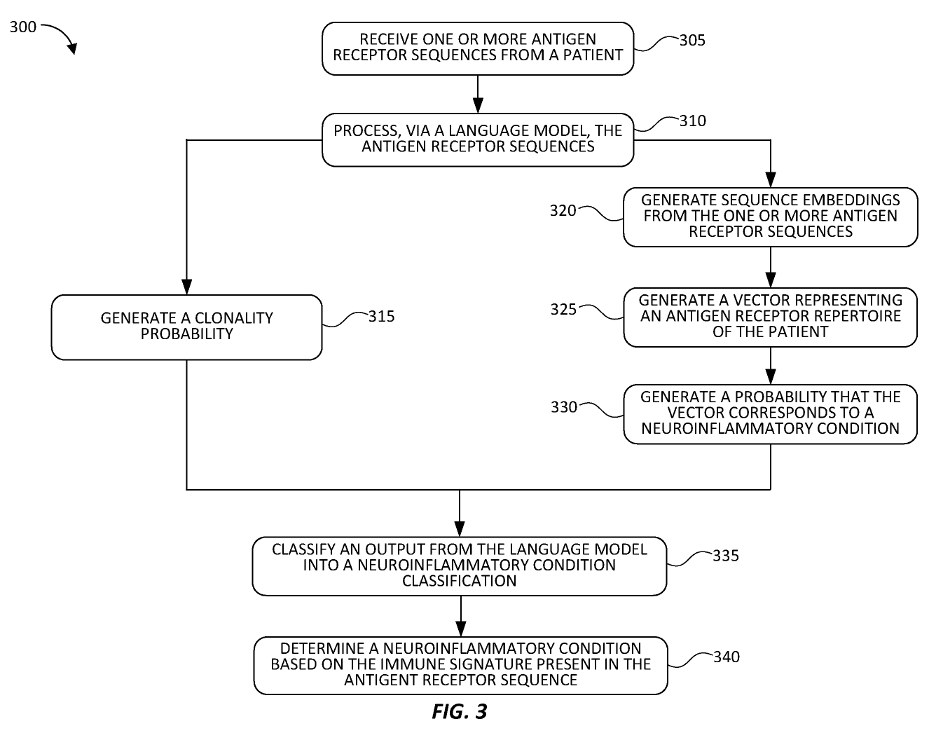

Referring now to FIG. 2, an example IS detection engine 216 is provided, according to an embodiment herein. For ease of illustration, FIG. 2 is described with respect to FIG. 3, which provides a process 300 for providing an IS detection engine and its related functions, such as the IS detection engine 216, according to an embodiment herein. Although FIG. 3 is described in relation to FIG. 2, it should be appreciated that the process 300 of FIG. 3 is equally applicable to the remaining Figures and components therein.

To generate a prediction of a patient’s neuroinflammatory condition or disease status, such as a prediction of patient’s 102 neuroinflammatory condition or disease status, the IS detection engine 216 may receive one or more antigen receptor sequences 208 from a client device 210 (305). The client device 210 may be the same or similar to the client device 110 and, as such, be associated with a provider. The antigen receptor sequences 208 may be adaptive immune receptor sequences extracted from a blood sample or CSF sample from the patient 102. In example embodiments, the antigen receptor sequences 208 contain BCR IGH sequences, IGL/K sequences, and/or TCRα/β sequences. For ease of explanation, the antigen receptor sequences 208 are BCR IGH sequences in the following example.

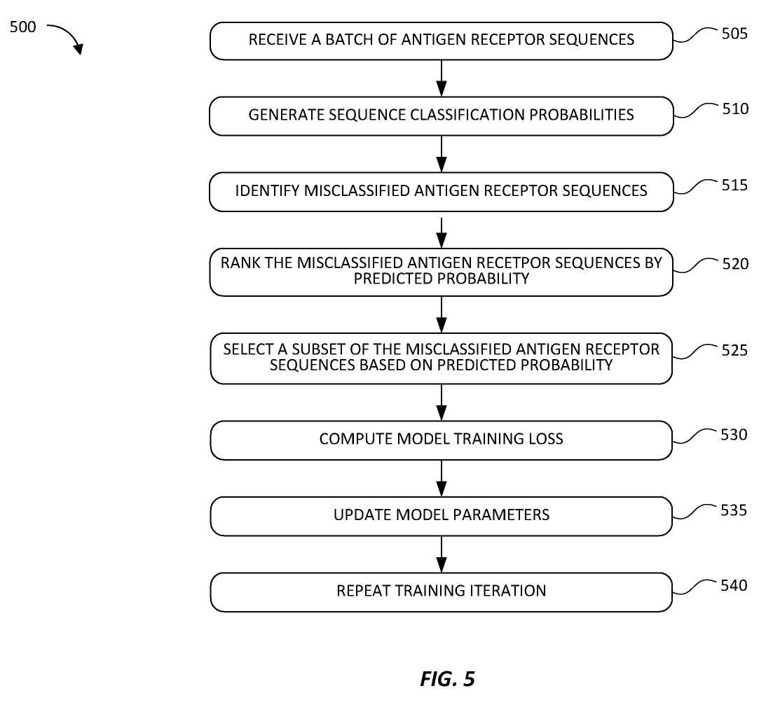

Responsive to receiving the antigen receptor sequences 208, the IS detection engine 216 processes the antigen receptor sequences 208 via one or more model(s) 222 (310). The one or more model(s) 222 may include a machine learning (ML) or artificial intelligence (AI) model that is trained to determine an immune signature present in the antigen receptor sequences 208. An example training routine used to train the one or more model(s) 222 is described in greater detail below with respect to FIG. 5.

In some embodiments, the one or more model(s) 222 may include a DNA language model configured to process nucleotide sequences in a manner analogous to natural language processing (NLP) large language models using transformer architectures. In such embodiments, the model 222 may be configured to identify probabilistic relationships between nucleotide sequences, including antigen receptor sequences 208, and respective neuroinflammatory conditions. In some embodiments, the DNA language model may be implemented as an autoregressive language model in which sequence elements are predicted sequentially based on previously observed elements. For example, the DNA language model may include a transformer-decoder architecture that utilizes self-attention mechanisms and feed-forward layers to model long-range dependencies within antigen receptor sequences 208. In certain implementations, the DNA language model may include a transformer-decoder model comprising multiple layers and hundreds of millions of trainable parameters.

Other examples of autoregressive language models that may be leveraged by the IS detection engine 216 to process antigen receptor sequences include GPT-based architectures, such as GPT-2, GPT-3, or GPT-4, which utilize multi-head self-attention and positional embeddings to capture sequence dependencies. Additionally, models such as XLNet, which incorporate permutation-based training objectives, or Reformer architectures that employ locality-sensitive hashing (LSH) for efficient attention computation, may also be leveraged to enhance scalability and performance when processing long antigen receptor sequences.

In some embodiments, the DNA language model implemented within the model 222 may include a decoder-only transformer-based language model trained to process antigen receptor sequences. For example, the model 222 may be pre-trained using next-token prediction tasks or other sequence modeling objectives in which the model 222 predicts subsequent nucleotide tokens based on preceding tokens within the sequence. Such pre-training enables the model 222 to learn contextual relationships among nucleotide sequences associated with antigen receptors and facilitates identification of immune signatures within antigen receptor repertoires.

In certain example implementations, multiple variants of the language foundation model may be trained with different model sizes. For instance, large, medium, and small model variants may be trained using similar training objectives but differing numbers of trainable parameters. Performance of the model variants may be evaluated to determine an appropriate model configuration for downstream immune signature detection tasks.

In some embodiments, pretraining data used to train the model 222 may include antigen receptor sequences obtained from immune receptor repositories or other sequence datasets. For example, B-cell receptor IGH sequences may be extracted and processed to generate a diverse training dataset. In certain implementations, sequence fragments or token representations, such as k-mer representations (e.g., 7-mers), may be generated from the antigen receptor sequences to facilitate language-model-based training.

In some embodiments, the training dataset may be curated to increase sequence diversity. For example, sequences may be sampled based on similarity metrics, such as distances in k-mer representation space, to construct a compact dataset containing diverse antigen receptor sequences. Such sampling may reduce redundancy in the dataset while preserving representative sequence diversity relevant to immune receptor repertoires.

During pretraining, antigen receptor sequences may be tokenized at the nucleotide level and processed using augmentation or masking strategies. For example, sequences may be randomly rotated, masked, or truncated to encourage the model 222 to learn generalized sequence features. In certain implementations, attention masks may be applied to subsets of nucleotides to simulate incomplete sequence observations and improve robustness of the resulting sequence representations.

In some embodiments, the model 222 may generate sequence-level probability outputs indicating whether individual antigen receptor sequences are associated with particular disease categories. For example, a probability output layer may be applied to hidden-state representations generated by the language model. In certain implementations, the prediction layer may be connected to hidden-state representations associated with the final token of an antigen receptor sequence, enabling the model 222 to generate probability scores indicating the likelihood that a given sequence corresponds to a particular disease category.

During training of the model 222, antigen receptor sequences may be labeled according to disease status associated with the patient from which the sequences were obtained. For example, patient disease labels may be propagated to the corresponding antigen receptor sequences and used to assign sequences to disease-related categories such as healthy, autoimmune disease, infectious disease, or neuroinflammatory disease categories including multiple sclerosis. In some embodiments, subsets of antigen receptor sequences from each patient may be sampled to construct the training dataset.

In certain implementations, the training dataset may be partitioned into multiple folds to facilitate model evaluation and reduce potential bias associated with study-specific or dataset-specific features. For example, antigen receptor sequences originating from the same study or dataset may be grouped together and assigned to the same fold during training and validation procedures.

Training of the model 222 may be performed using gradient-based optimization techniques. For example, a cross-entropy loss function may be used to optimize sequence probability outputs generated by the model 222. Learning rate schedules may be applied during training, and gradient accumulation may be used to increase the effective batch size used for parameter updates. In some embodiments, domain-specific loss strategies may be applied to improve detection of disease-associated antigen receptor sequences. For example, during certain training iterations a portion of sequences assigned to disease-related categories may be temporarily relabeled as healthy for purposes of calculating the loss function. Such relabeling may reduce the influence of dataset-specific biases and encourage the model 222 to identify sequence features strongly associated with disease-related immune signatures.

In certain implementations, the model 222 may be trained using any suitable computing infrastructure, including graphics processing units (GPUs), tensor processing units (TPUs), or other parallel processing systems capable of efficiently processing large volumes of antigen receptor sequence data. Performance of different model configurations may be compared based on their ability to generate embeddings or sequence predictions that capture immune signatures associated with neuroinflammatory conditions, and a selected model configuration may be used for subsequent embedding generation and classification tasks performed by the IS detection engine 216. An example training routine for training the model 222 is described below with respect to FIG. 5.

As noted above, in some embodiments, the IS detection engine 216 may be leveraged to determine whether the patient has a particular neuroinflammatory condition, while in other embodiments, the IS detection engine 216 may be leveraged to determine a disease status of the patient for a known neuroinflammatory condition. In yet other embodiments, the IS detection engine 216 may be leveraged to determine both the neuroinflammatory condition of the patient and then the disease status of the neuroinflammatory condition. For ease of explanation, the following discussion initially focuses on the IS detection engine 216 detecting a neuroinflammatory condition for the patient and then on the IS detection engine 216 determining a disease status of the neuroinflammatory condition for the patient. Although the following processes are described separately, it should be appreciated that in some embodiments, the processes may be performed simultaneously, sequentially, or in parallel to one another.

To detect the neuroinflammatory condition or determine the disease status of the patient, the IS detection engine 216 may process the antigen receptor sequences 208 via the one or more model(s) 222 to generate an immune signature (310). The immune signature, as used herein, may reflect the clonality of antigen receptors within the antigen receptor sequences 208 or may represent the overall antigen receptor repertoire relationships within the antigen receptor sequences 208. Clonality of the antigen receptors within the antigen receptor sequences 208 includes both public clonality and private clonality of antigen receptors. Public clonality of antigen receptors refers to antigen receptor sequences that are shared among multiple individuals within a given population, suggesting a common immune response to a specific antigenic stimulus. Private clonality, in contrast, refers to antigen receptor sequences that are unique to an individual, reflecting personalized immune responses driven by individual antigen exposure or genetic factors. For example, in the case of BCRs and a neuroinflammatory condition such as MS, public clonality may include antigen receptor sequences that are recurrently observed across different MS patients, potentially indicative of a common autoantigen-driven immune response. Private clonality, on the other hand, may encompass unique BCR sequences specific to an individual’s immune history, influenced by personal antigenic encounters and immune selection pressures.

To determine the neuroinflammatory condition of the patient, model 222 processes the antigen receptor sequences 208 to extract numerically interpretable features within the antigen receptor sequences 208. These extracted sequence features or feature relationships within the antigen receptor sequences 208 may be referred to herein as immune adaptive receptors. Specifically, the model 222 may include an autoregressive language model that generates a numerical probability that clonotypes within the antigen receptor sequences 208 correspond to a neuroinflammatory condition (315). Following the above MS example, the model 222 may process the antigen receptor sequences 208 and identify sequence features or relationships between antigen receptor sequences 208 (e.g., immune adaptive receptors) associated with MS. As expanded on below, the model 222 may be trained via a training dataset containing antigen receptor sequences 208 representative of sequence features or features relationships recurrent across different MS patients. Similarly, the model 222 may process the antigen receptor sequences 208 to identify private clonotypes within the antigen receptor sequences 208 associated with MS. Since private clonotypes of MS are unique to individual patients and public clonotypes of MS may vary across different MS populations, the model 222 generates a probability or confidence score that the detected sequences are associated with MS as an output 224.

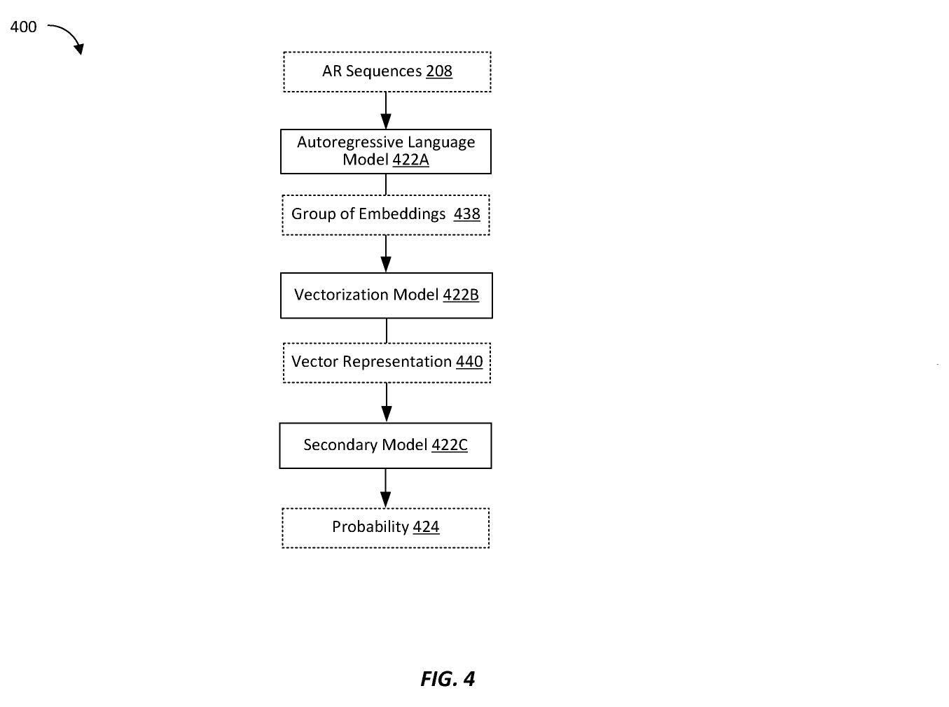

In some embodiments, to determine the neuroinflammatory condition and/or a disease status of a respective neuroinflammatory condition, the model(s) 222 may process the antigen receptor sequences 208 to generate a vector representation representing an antigen receptor repertoire of the patient. Reference is now made to FIG. 4 which illustrates an example operational environment 400 of the model(s) 222 determining a disease status of a patient, according to an embodiment herein. The environment 400 provides an illustrative example of the model(s) 222, including various inputs and outputs between the model(s) 222. As shown, to determine a disease status, the model(s) 222 may include an autoregressive model 422A, vectorization model 422B, and a secondary classification model 422C.

As shown, the antigen receptor sequences 208 are submitted as an input into an autoregressive language model 422A, which may be a transformer-decoder language model. Responsive to receiving the antigen receptor sequences 208, the autoregressive language model 422A generates a group of embeddings 438 from the antigen receptor sequences 208 (320), where an antigen receptor sequence 208 is projected into a respective embedding 438. In an example embodiment, the antigen receptor sequences 208 are processed by the autoregressive language model 422A and the embeddings 438 are extracted from hidden states prior to a final model layer of the autoregressive language model 422A. The embeddings 438 may include 1024 (or another integer) values, representative of each antigen receptor sequence 208.

The embeddings 438 are sequence embeddings that include a numerical representation of a biological sequence generated by a machine learning model, such as a language model within the model(s) 222. The sequence embedding may be represented as a vector of numerical values that encodes features, patterns, or relationships present within the biological sequence. In certain implementations, sequence embeddings are generated from antigen receptor sequences and positioned within an embedding space such that sequences having similar structural, functional, or immunological characteristics are represented by vectors located in similar regions of the embedding space. Sequence embeddings may be used as inputs to downstream models or processing steps to analyze relationships among antigen receptor sequences, generate repertoire-level representations, or determine immune signatures associated with particular biological conditions.

The group of embeddings 438 may then be submitted to a vectorization model 422B. The vectorization model 422B generates a lower dimensional vector representation 440 of the group of embeddings 438, representing the immune receptor repertoire (325). For instance, the vectorization model 422B may calculate a local density of adaptive immune receptors, as represented by the group of embeddings 438, around a defined number of waypoints in the embedding space. The local density of the group of embeddings 438 may then be used to generate a single vector representation 440 of the embeddings 438. The vectorization model 422B may output the vector representation 440 to the secondary classification model 422C.

The secondary classification model 422C may receive the vector representation 440 as an input. The secondary classification model 422C, which may be a convolutional neural network classifier or random forest classifier, may generate a probability that the repertoire derived from the patient, as represented by the vector representation 440, corresponds to a particular neurological disorder (330). In some embodiments, the probability 424 output by the secondary classification model 422C is to a particular disease status of the patient, depending on the configuration of the secondary classification model 422C. It should be noted that while the operational environment 400 of the model(s) 222 is described as determining a disease status of a neuroinflammatory condition, in some embodiments, one or more of the above steps and/or components may be used to detect the neuroinflammatory condition.

In some embodiments, prior to submitting the antigen receptor sequences 208 to the model(s) 222, the IS detection engine 216 may preprocess the antigen receptor sequences 208 to enhance accuracy and interpretability. Preprocessing of the antigen receptor sequences 208 may include quality filtering to remove sequencing artifacts such as low-quality reads, adapter sequences, and PCR duplicates, thereby improving downstream analysis reliability. Additionally, preprocessing may involve aligning the antigen receptor sequences 208 to a reference BCR or TCR database using specialized bioinformatics tools (e.g., IgBlast, MiXCR, or IMGT/HighV-QUEST) to annotate their variable (V), diversity (D), and joining (J) gene segment usage. This pre-processing enables accurate identification of clonotypes and provides insights into repertoire diversity. Furthermore, preprocessing may include identifying and characterizing the complementary-determining region 3 (CDR3), which is critical for antigen specificity. By standardizing and refining the antigen receptor sequences 208 through preprocessing, the IS detection engine 216 can efficiently process sequence quality, repertoire diversity, clonal expansion, and antigen specificity, thereby enhancing the accuracy of the model(s) 222 predictions.

In some embodiments, to accurately and confidently diagnose a patient, IS detection engine 216 may require a threshold number of adaptive immune receptors within the antigen receptor 208. For example, to accurately detect MS or determine a diseases state of MS, the IS detection engine 216 may require a minimum threshold of 1,000 unique BCR IGH sequences (e.g., private clonotypes) within the antigen receptor sequences’ 208 repertoire. In some cases, the minimum threshold may be 2,000 unique clonotypes, 5,000 clonotypes, or 10,000 clonotypes within a respective patient’s repertoire.

As noted above the model(s) 222 may be trained to determine an immune signature within the antigen receptor sequences 208 and generate a probability that the identified immune signature corresponds to a particular neuroinflammatory condition. As shown, to train the model(s) 222, the IS detection engine 216 may be in operable communication with a trainer 242. The trainer 242 may train the model(s) 222 based on a training dataset 244. As shown, the training dataset 244 may include historical antigen receptor sequences 246 and corresponding labels 248. The historical antigen receptor sequences 246 may include a group first of antigen receptor sequences corresponding to one or more neuroinflammatory conditions and a second group of antigen receptor sequences corresponding to healthy controls. In such cases, the labels 248 may include corresponding diseased labels 250 and healthy labels 252. The trainer 242 may use the training dataset 244 to train the model(s) 222 to identify immune signatures within antigen receptor sequences and predict whether the immune signatures correspond to a neuroinflammatory condition or a healthy control.

During training, the model 222 processes batches of the historical antigen receptor sequences 246 by passing the sequences through one or more model layers (e.g., transformer-decoder layers) to generate sequence-level probability outputs indicative of immune signatures and associated disease categories. The model 222 computes a difference between the generated probability outputs and the corresponding labels 248 to determine a training loss. Using backpropagation, gradients of the training loss are determined with respect to model parameters and the model parameters are updated based on the gradients to improve model performance over multiple training iterations and/or training epochs. In some embodiments, the trainer 242 may employ one or more gradient-based optimization techniques, such as stochastic gradient descent (SGD), Adam, AdamW, or combinations thereof, and in certain implementations may use distributed training in which gradients and/or parameter updates are aggregated across multiple computing nodes.

The trainer 242 may leverage the HPC cluster to ensure high-speed data transfer between nodes and parallel execution of tasks, making the training process much faster and more efficient than on a single machine. Throughout training, performance metrics like accuracy and loss are monitored, and the model’s 222 weights are periodically saved for checkpointing. Once trained, the model 222 can then predict whether the submitted antigen receptor sequences 208 correspond to a neuroinflammatory condition or a healthy state, or in some cases, what neuroinflammatory condition from a grouping of neuroinflammatory conditions the antigen receptor sequences 208 correspond. In some embodiments, to enhance training efficiency, the trainer 242 leverages a high-performance computing (HPC) cluster, enabling high-speed data transfer and parallel execution across multiple nodes. Throughout training, key performance metrics such as accuracy and loss are continuously monitored to evaluate model improvement. Additionally, model 222 weights are periodically checkpointed to preserve training progress and facilitate recovery if needed.

The trainer 242 may leverage an IS training routine to train the model 222. The IS training routine may relax how the trainer 242 backpropagates error during training based on the application. That is, the trainer 242 allows the model 222 to misclassify a certain rate of the historical antigen receptor sequences 246 without penalty (e.g., 7 out 8 errors before a penalty is applied). If the model 222 is trained without the IS training routine, the model 222 may generate an undesirable amount of false positives. For example, because patients having neuroinflammatory conditions inherently include both diseased antigen receptor sequences and healthy antigen receptor sequences within their repertoire, if the model 222 is forced to apply the diseased label 250 to each antigen receptor sequence of the diseased patient (such as under conventional training routines), the model 222 will start applying the diseased label 250 to other healthy antigen receptor sequences, including those corresponding to the healthy controls. This results in an increase of false positives. To prevent this, the trainer 242 minimizes the penalty of misclassification by the model 222.

Referring now to FIG. 5, an example IS training routine 500 is illustrated, according to an embodiment herein. The IS training routine 500 may be used by the trainer 242 to train the model 222. It should be appreciated that the IS training routine 500 illustrates a single training iteration and the steps described below may be performed in an iterative manner until the model 222 reaches a desirable degree of accuracy and recall. In particular, the IS training routine 500 illustrates an example workflow in which antigen receptor sequences are processed to generate sequence classification probabilities, misclassified sequences are identified and ranked based on predicted probability, and a subset of the misclassified sequences is used to determine a model training loss and update parameters of the model 222.

During the training process, the trainer 242 may receive a batch of antigen receptor sequences (505) from the training dataset 244. For example, a subset of the historical antigen receptor sequences 246 may be processed by the model 222. For the subset of historical antigen receptor sequences 246, which includes both diseased antigen receptor sequences and healthy antigen receptor sequences, the model 222 may determine the immune signature and generate sequence classification probabilities (510) indicating the likelihood that the respective historical antigen receptor sequence 246 corresponds to either a neuroinflammatory condition or a healthy control.

After the model 222 processes the batch of antigen receptor sequences, the trainer 242 identifies misclassified antigen receptor sequences (515). For example, the trainer 242 may determine a group of diseased antigen receptor sequences that are misclassified as healthy controls or healthy antigen receptor sequences that are misclassified as diseased sequences. The trainer 242 then ranks the misclassified antigen receptor sequences by predicted probability (520). That is, the trainer 242 ranks the misclassified antigen receptor sequences according to the confidence scores (e.g., probabilities) generated by the model 222 based on the immune signature of the underlying antigen receptor sequence.

From the ranking, the trainer 242 then selects a subset of the misclassified antigen receptor sequences based on the predicted probability (525). For example, the trainer 242 may select a subset of misclassified antigen receptor sequences having particular probability characteristics, such as sequences associated with relatively high or relatively low confidence predictions. The selected subset of misclassified antigen receptor sequences may then be used to compute a model training loss (530). By determining the loss using the selected subset of misclassified antigen receptor sequences, the IS training routine 500 may adjust how particular sequences influence the training process. For example, certain misclassified sequences that are difficult to classify or that may not be strongly associated with a disease-related immune signature may contribute differently to the training loss. In some embodiments, the IS training routine 500 may include other approaches and techniques, such as gradient relaxation, where instead of completely excluding sequences from the loss calculation, the model 222 adjusts or down-weights their contribution to the loss function.

After the model training loss is computed, the trainer 242 updates parameters of the model 222 (535). For example, the model parameters may be updated using gradient-based optimization techniques such as backpropagation to improve the model's ability to distinguish immune signatures associated with neuroinflammatory conditions from those associated with healthy controls. Following the parameter update, the IS training routine 500 may repeat the training iteration (540), such that additional batches of antigen receptor sequences from the training dataset 244 are processed to further refine the model 222.

Returning now to FIG. 2, once the model(s) 222 process the antigen receptor sequences 208 to determine an immune signature and generate sequence-level probability outputs, the output 224, which may include one or both of the clonal probabilities or the probability 424 that the repertoire derived from the patient indicates a particular neuroinflammatory condition or disease status, may be provided directly to a classifier 226 or may be further processed to generate repertoire-level representations prior to classification. In some embodiments, the classifier 226 may utilize one or more machine learning models configured to classify patient repertoires based on sequence-derived features or repertoire-level embeddings. Such models may include logistic regression models, ensemble models, random forest models, gradient boosting models, support vector machines (SVMs), neural networks, or other supervised learning techniques. These models may operate individually or in combination as an ensemble to improve classification accuracy and robustness.

In some embodiments, the antigen receptor sequence outputs generated by the model 222 may be aggregated to generate a repertoire-level representation corresponding to a patient. For example, embeddings associated with individual antigen receptor sequences may be compared to a set of reference vectors within an embedding space derived from the model 222. Distances or similarity measures between the sequence embeddings and the reference vectors may be determined to characterize the distribution of sequences within the patient’s antigen receptor repertoire. In certain implementations, the resulting similarity or distance values may be aggregated across the antigen receptor sequences associated with the patient to generate a repertoire-level embedding or feature vector representing the immune repertoire of the patient. The repertoire-level representation may then be provided as input to the classifier 226 to facilitate classification of the patient with respect to a neuroinflammatory condition or disease status.

Responsive to receiving the probabilities generated by the model(s) 222 via the output 224, the classifier 226 processes the probabilities assigned to the antigen receptor sequences 208 and/or repertoire-level representations derived from the antigen receptor sequences to generate a classification 232 (335). For instance, based on the clonal probabilities generated by the model(s) 222, the classifier 226 classifies the antigen receptor sequences 208 into the classification 232. The probabilities represent confidence scores that quantify the likelihood that the antigen receptor sequences 208 exhibit a particular immune signature identified by the model(s) 222. As such, to classify the antigen receptor sequences 208, the classifier 226 may apply a predefined threshold to the probability received from the model(s) 222, wherein antigen receptor sequences 208 exceeding the threshold are assigned to a specific classification, while elements below the threshold may be assigned to an alternative classification. In cases where multiple classification models are utilized, the classifier 226 may aggregate the probabilities using techniques such as weighted averaging, majority voting, or Bayesian inference to improve classification reliability.

As shown, the classifier 226 may classify the antigen receptor sequences 208 into different classifications, such as a neuroinflammatory condition classification 228 and a healthy classification 230. Depending on the configuration of the IS detection engine 216, the classifier 226 may include only a neuroinflammatory condition classification 228 and the healthy classification 230, such as classifying the antigen receptor sequences 208 into either an MS classification (neuroinflammatory condition classification 228) or the healthy classification 230.

In other embodiments, the IS detection engine 216 may be configured to distinguish between different neuroinflammatory conditions, and as such, the classifier 226 may classify the antigen receptor sequences 208 into one of a group of neuroinflammatory condition classifications 228A-n. For example, the classifier 226 may be able to determine that the immune signature present in the antigen receptor sequences 208 is indicative of a neuroinflammatory condition, and classify the antigen receptor sequences 208 into a particular neuroinflammatory condition classification 228A-n based on the probabilities present in the output 224.

In yet another embodiment, the neuroinflammatory condition classifications 228A-n may indicate a disease status instead of differentiating between different types of neuroinflammatory conditions. For example, the neuroinflammatory condition classification 228A may indicate earlier stages of a respective neuroinflammatory condition, as detected by the immune signature within the antigen receptor sequences 208, while the subsequent neuroinflammatory condition classifications 228B-n may indicate later stages of the neuroinflammatory condition, respectively.

Once the classifier 226 processes the output 224, the antigen receptor sequences or embeddings 208 may be classified into an appropriate classification 232. Responsive to the classification 232, the IS detection engine 216 may determine the neuroinflammatory condition based on the immune signature present in the antigen receptor sequences 208 (340). In particular, the IS detection engine 216 may include a neuroinflammatory condition detector 234. The classification 232 is provided to the neuroinflammatory condition detector 234 which in turn determines a neuroinflammatory condition 236A or a disease status 236B of the patient. The neuroinflammatory condition detector 234 determines the neuroinflammatory condition 236A based on the classification 232 of the antigen receptor sequences 208, and/or similarly determines the disease status 236B based on the classification 232.

The neuroinflammatory condition detector 234 may generate a prediction 218, which may be the same or similar to the predicted diagnosis 118. The prediction 218 may provide a predicted diagnosis 118 of the neuroinflammatory condition 236A and/or the disease status 236B for the patient based on the antigen receptor sequence 208. As illustrated, the prediction 218 may be transmitted by the IS detection engine 216 to the client device 210, such as via the application service 112. The prediction 218 may be provided to a provider, such as displayed on the user interface 120. Using the prediction 218, the provider can determine an official diagnosis of the patient, and the provider and the patient can make an informed decision about the best treatment route for the patient.

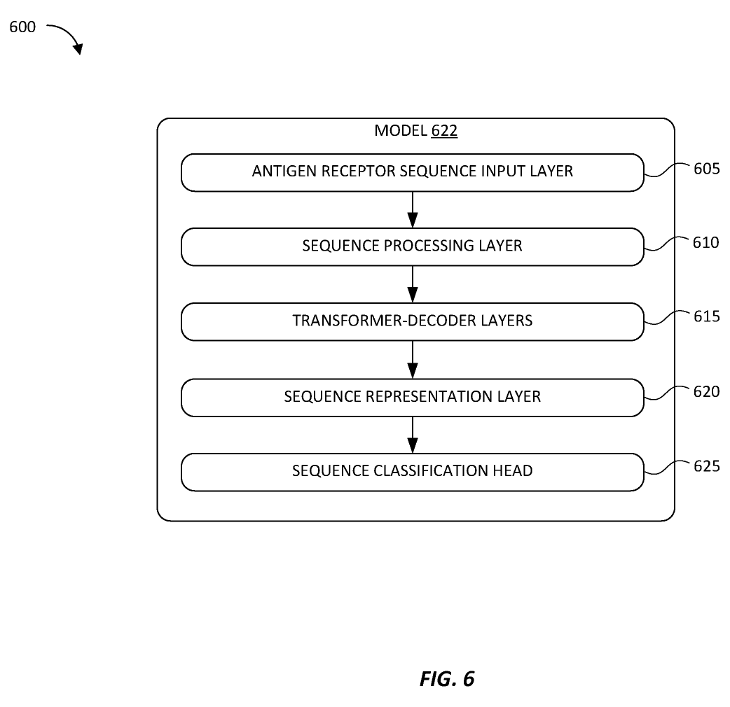

Referring now to FIG. 6, an example architecture 600 of a model 622 is illustrated, according to embodiments described herein. As shown, the model 622, which may be the same or similar to the model 222, is configured to receive antigen receptor sequences and generate sequence-level probability outputs indicative of immune signatures associated with neuroinflammatory conditions.

In some embodiments, the model 622 includes an antigen receptor sequence input layer 605 configured to receive antigen receptor sequences. For example, the antigen receptor sequences may include adaptive immune receptor sequences obtained from a patient sample, such as BCR or TCR sequences. In certain implementations, the antigen receptor sequences may include nucleotide sequences corresponding to IGH, immunoglobulin light chain (IGL or IGK), or T-cell receptor α/β sequences.

Upon receipt, the antigen receptor sequences are provided to a sequence processing layer 610. In some embodiments, the sequence processing layer 610 may perform preprocessing operations on the antigen receptor sequences prior to processing by the model architecture. Such preprocessing operations may include tokenization of nucleotide sequences, sequence normalization, sequence masking, augmentation of sequence inputs, or other preprocessing techniques configured to prepare the antigen receptor sequences for input to the model 622.

In the illustrated example, the model 622 includes one or more transformer-decoder layers 615 that process the antigen receptors after preprocessing. In some embodiments, the transformer-decoder layers 615 may include multiple stacked transformer layers configured to learn contextual relationships among nucleotide tokens within the antigen receptor sequences. For example, the transformer-decoder layers 615 may utilize self-attention mechanisms and feed-forward neural network layers to model long-range dependencies within the antigen receptor sequences. Through this processing, the transformer-decoder layers 615 may generate contextualized sequence representations capturing relationships between sequence elements associated with immune signatures.

Within the model 622, outputs of the transformer-decoder layers 615 are provided to a sequence representation layer 620. In some embodiments, the sequence representation layer 620 generates embedding vectors or other numerical representations corresponding to the antigen receptor sequences processed by the transformer-decoder layers. These sequence representations may encode features or relationships present within the antigen receptor sequences that are associated with immune responses or disease-related immune signatures.

The sequence representations generated by the sequence representation layer 620 are then provided to a sequence classification head 625. In some embodiments, the sequence classification head 625 may include a prediction layer configured to generate sequence-level probability outputs for the antigen receptor sequences. For example, the sequence classification head 625 may produce probability values indicating whether a given antigen receptor sequence is associated with a particular disease category, immune signature, or neuroinflammatory condition.

The sequence-level probability outputs generated by the sequence classification head 625 may be provided as part of the output of the model 622, such as the output 224. As described above, the output 224 may be used by downstream components of the IS detection engine 216, such as the classifier 226, to determine a neuroinflammatory condition or disease status associated with the patient.

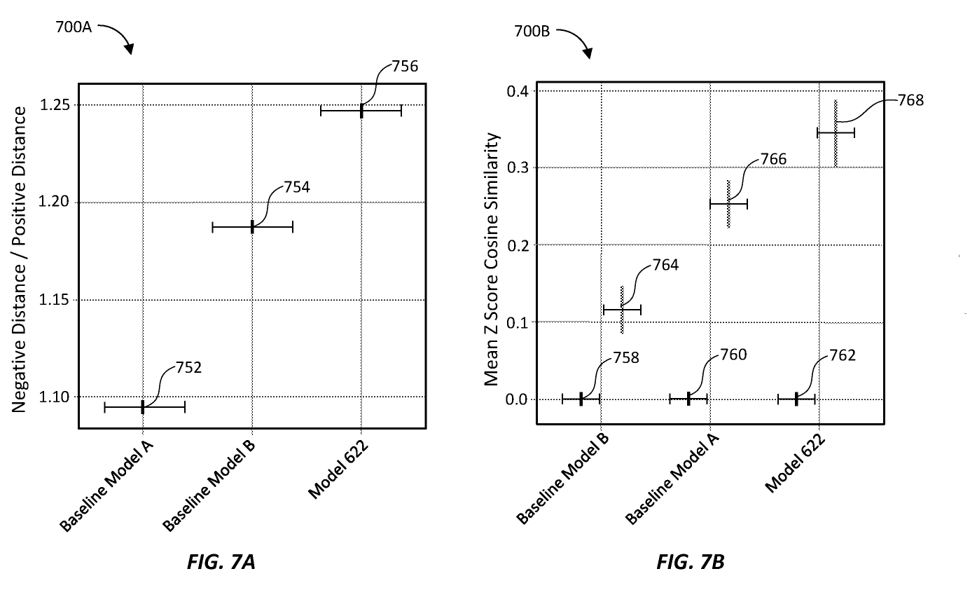

Referring now to FIGS. 7A and 7B, comparative evaluations of sequence embedding models are illustrated. In particular, FIG. 7A illustrates a first graph 700A showing a separation metric between disease-associated and non-disease-associated antigen receptor sequences, while FIG. 7B illustrates a second graph 700B showing a similarity metric for antigen receptor sequence embeddings generated by different sequence embedding models. The graphs 700A and 700B are generated by evaluating embeddings produced by the disclosed model 622 relative to baseline embedding models. As described herein, the embeddings generated by the model 622 provide improved structural organization of antigen receptor sequences within an embedding space, thereby improving the ability of downstream components of the IS detection engine 216 to detect immune signatures associated with neuroinflammatory conditions.

In the illustrated example, the baseline models shown in FIGS. 7A and 7B include transformer-based protein or nucleotide sequence embedding models used as reference systems. Baseline Model A corresponds to a general-purpose protein language model configured to generate embeddings for biological sequences, while Baseline Model B corresponds to a transformer-based antibody or immune-repertoire language model trained on antigen receptor sequences. These baseline models serve as representative reference models for evaluating embedding performance relative to the disclosed model 622.

Referring to FIG. 7A, the graph 700A illustrates a negative-distance-to-positive-distance ratio generated from embeddings produced by the evaluated models. In this context, “positive” distances correspond to distances between embeddings associated with antigen receptor sequences that share a similar immune signature or disease classification, while “negative” distances correspond to distances between embeddings associated with antigen receptor sequences having different immune signatures or disease classifications. The ratio of negative distance to positive distance indicates how effectively the embedding space separates sequences associated with different immune conditions, with larger ratios indicating improved separation between disease-associated and non-disease-associated antigen receptor sequences.

As shown in graph 700A, a first indicator 752 corresponds to Baseline Model A and is positioned near a ratio value below approximately 1.10. A second indicator 754 corresponds to Baseline Model B and is positioned near a ratio value below approximately 1.20. A third indicator 756 corresponds to the disclosed Model 622 and is positioned near a ratio value of approximately 1.25. The relative positions of the indicators 752, 754, and 756 demonstrate that the embeddings generated by Model 622 exhibit a larger negative-to-positive distance ratio than embeddings generated by Baseline Model A and Baseline Model B. This increased ratio indicates that Model 622 produces greater separation in embedding space between sequences associated with different immune signatures while maintaining comparatively smaller distances among sequences associated with similar immune signatures.

The improved separation illustrated by the indicator 756 in graph 700A reduces overlap between disease-associated and non-disease-associated antigen receptor sequences within the embedding space. As a result, embeddings generated by Model 622 provide improved feature representations for downstream immune signature detection and patient classification, including classification performed by the classifier 226 to determine a neuroinflammatory condition or disease status.

Referring now to FIG. 7B, the graph 700B illustrates a mean Z-scored cosine similarity metric generated from embeddings produced by the evaluated models. Cosine similarity measures the similarity between embedding vectors corresponding to antigen receptor sequences. In the illustrated graph 700B, cosine similarity values are normalized using Z-score normalization to enable comparison of similarity values generated by different embedding models.

As shown in graph 700B, a first set of indicators 758, 760, and 762 correspond to similarity measurements calculated between embeddings associated with antigen receptor sequences belonging to different immune signatures or disease classifications. In particular, indicator 758 corresponds to Baseline Model B, indicator 760 corresponds to Baseline Model A, and indicator 762 corresponds to the disclosed Model 622. Each of the indicators 758, 760, and 762 is positioned approximately at a similarity value of 0.0, indicating that embeddings generated by each of the evaluated models exhibit minimal similarity between sequences associated with different immune signatures.

A second set of indicators 764, 766, and 768 correspond to similarity measurements calculated between embeddings associated with antigen receptor sequences sharing the same immune signature or disease classification. In particular, indicator 764 corresponds to Baseline Model B and is positioned at approximately 0.15, indicator 766 corresponds to Baseline Model A and is positioned at approximately 0.25, and indicator 768 corresponds to the disclosed Model 622 and is positioned at approximately 0.34. The relative positions of indicators 764, 766, and 768 demonstrate that embeddings generated by Model 622 produce stronger similarity among sequences associated with the same immune signature relative to the baseline models.

The combination of indicators shown in graph 700B demonstrates that Model 622 maintains low similarity between sequences associated with different immune signatures (indicators 758–762) while simultaneously increasing similarity among sequences associated with the same immune signature (indicators 764–768). This improved clustering behavior within the embedding space allows antigen receptor sequences associated with similar immune responses to be grouped more tightly while maintaining separation from sequences associated with different immune responses.

As a result, the embeddings generated by Model 622 provide improved structural organization of antigen receptor sequences within the embedding space. This improved organization enhances the ability of downstream components of the IS detection engine 216, including the classifier 226, to more accurately distinguish between antigen receptor repertoires associated with neuroinflammatory conditions and repertoires associated with healthy controls.

Referring to FIG. 8, FIG. 8 illustrates a computing apparatus 891 that may be used for providing an IS detection engine and related functions, as described herein. For example, the client devices 110 or 210, or the server computers 114 may be or include the computing apparatus 891. As illustrated, the computing apparatus 891 includes a processing system 892 that includes a microprocessor and other circuitry that retrieves and executes software 895 from storage system 893. The processing system 892 may be implemented within a single processing device but may also be distributed across multiple processing devices or sub-systems that cooperate in executing program instructions. Examples of the processing system 892 include general purpose central processing units, graphical processing units, application specific processors, and logic devices, as well as any other type of processing device, combinations, or variations thereof.

The storage system 893 may comprise any computer-readable storage media or medium readable by processing system 892 and capable of storing software 895. The storage system 893 may include volatile and nonvolatile, removable and non-removable media implemented in any method or technology for storage of information, such as computer readable instructions, data structures, program modules, or other data. Examples of storage media include random access memory, read only memory, magnetic disks, optical disks, flash memory, virtual memory and non-virtual memory, magnetic cassettes, magnetic tape, magnetic disk storage or other magnetic storage devices, or any other suitable storage media. In no case is the computer readable storage media a propagated signal.

In addition to computer readable storage media, in some implementations the storage system 893 may also include computer readable communication media over which at least some of the software 895 may be communicated internally or externally. The storage system 893 may be implemented as a single storage device but may also be implemented across multiple storage devices or sub-systems co-located or distributed relative to each other. The storage system 893 may comprise additional elements, such as a controller capable of communicating with the processing system 892 or possibly other systems.

The software 895 (including IS detection engine process 896) may be implemented in program instructions and among other functions may, when executed by the processing system 892, direct the processing system 892 to operate as described with respect to the various operational scenarios, sequences, and processes illustrated herein. For example, the software 895 may include program instructions for implementing an IS detection engine and related functions, such as the process 300, or program instructions for training the IS detection engine via a training routine, such as implementing the IS training routine 500, as described herein.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel, in a single threaded environment or multi-threaded, or in accordance with any other suitable execution paradigm, variation, or combination thereof. The software 895 may include additional processes, programs, or components, such as operating system software, virtualization software, or other application software. The software 895 may also comprise firmware or some other form of machine-readable processing instructions executable by the processing system 892.

In general, the software 895 may, when loaded into the processing system 892 and executed, transform a suitable apparatus, system, or device (of which computing apparatus 891 is representative) overall from a general-purpose computing system into a special-purpose computing system customized to generate features, functionality, and user experiences provided by the IS detection engine. Indeed, encoding the software 895 on the storage system 893 may transform the physical structure of the storage system 893. The specific transformation of the physical structure may depend on various factors in different implementations of this description. Examples of such factors may include, but are not limited to, the technology used to implement the storage media of the storage system 893 and whether the computer-storage media are characterized as primary or secondary storage, as well as other factors.

For example, if the computer readable storage media are implemented as semiconductor-based memory, the software 895 may transform the physical state of the semiconductor memory when the program instructions are encoded therein, such as by transforming the state of transistors, capacitors, or other discrete circuit elements constituting the semiconductor memory. A similar transformation may occur with respect to magnetic or optical media. Other transformations of physical media are possible without departing from the scope of the present description, with the foregoing examples provided only to facilitate the present discussion.

Communication interface system 897 may include communication connections and devices that allow for communication with other computing systems (not shown) over communication networks (not shown). Examples of connections and devices that together allow for inter-system communication may include network interface cards, antennas, power amplifiers, RF circuitry, transceivers, and other communication circuitry. The connections and devices may communicate over communication media to exchange communications with other computing systems or networks of systems, such as metal, glass, air, or any other suitable communication media. The aforementioned media, connections, and devices are well known and need not be discussed at length here.

User interface system 899 may include various components and devices that enable interaction between the user and the computing system. Examples of these components and devices may include display screens, touchscreens, keyboards, mice, trackpads, styluses, voice recognition microphones, and other input/output devices. The user interface system 899 facilitates user commands and feedback through graphical user interfaces (GUIs), command-line interfaces (CLIs), or other interaction models. These interfaces may display information, receive user inputs, and provide visual, auditory, or tactile responses. The components and devices within the user interface system 899 are designed to ensure seamless and intuitive user interaction, leveraging well-established technologies and practices that need not be elaborated upon here.

Communication between the computing apparatus 891 and other computing systems (not shown), may occur over a communication network or networks and in accordance with various communication protocols, combinations of protocols, or variations thereof. Examples include intranets, internets, the Internet, local area networks, wide area networks, wireless networks, wired networks, virtual networks, software defined networks, data center buses and backplanes, or any other type of network, combination of network, or variation thereof. The aforementioned communication networks and protocols are well known and need not be discussed at length here.

While some examples of methods and systems herein are described in terms of software executing on various machines, the methods and systems may also be implemented as specifically-configured hardware, such as field-programmable gate array (FPGA) specifically to execute the various methods according to this disclosure. For example, examples can be implemented in digital electronic circuitry, or in computer hardware, firmware, software, or in a combination thereof. In one example, a device may include a processor or processors. The processor comprises a computer-readable medium, such as a random access memory (RAM) coupled to the processor. The processor executes computer-executable program instructions stored in memory, such as executing one or more computer programs. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example one or more non-transitory computer-readable media, which may store processor-executable instructions that, when executed by the processor, can cause the processor to perform methods according to this disclosure as carried out, or assisted, by a processor. Examples of may include, but are not limited to, an electronic, optical, magnetic, or other storage device capable of providing a processor, such as the processor in a web server, with processor-executable instructions. Other examples of non-transitory computer-readable media include, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code to carry out methods (or parts of methods) according to this disclosure.

Examples are described herein in the context of systems and methods for providing an IS detection engine and related functions. Those of ordinary skill in the art will realize that the foregoing description is illustrative only and is not intended to be in any way limiting. Reference is made in detail to implementations of examples as illustrated in the accompanying drawings. The same reference indicators will be used throughout the drawings and the following description to refer to the same or like items.

Additionally, the foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure. In the interest of clarity, not all of the routine features of the examples described herein are shown and described. It will, of course, be appreciated that in the development of any such actual implementation, numerous implementation-specific decisions must be made in order to achieve the developer’s specific goals, such as compliance with application- and business-related constraints, and that these specific goals will vary from one implementation to another and from one developer to another.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

### EXAMPLES

These illustrative examples are mentioned not to limit or define the scope of this disclosure, but rather to provide examples to aid understanding thereof. Illustrative examples are discussed above in the Detailed Description, which provides further description. Advantages offered by various examples may be further understood by examining this specification.

As used below, any reference to a series of examples is to be understood as a reference to each of those examples disjunctively (e.g., “Examples 1-4” is to be understood as “Examples 1, 2, 3, or 4”).

Example 1 is a computing apparatus comprising: a computer-readable storage medium; an immune signature (IS) detection engine comprising processor-executable instructions stored on the computer-readable storage medium; and a processor coupled to the computer-readable storage medium and configured to execute the processor-executable instructions, wherein the processor-executable instructions, when executed by the processor, direct the computing apparatus to at least: receive one or more antigen receptor sequences associated with a patient; identify an immune signature within the one or more antigen receptor sequences; generate a probability that the immune signature corresponds to one of a plurality of neuroinflammatory conditions; classify the one or more antigen receptor sequences into a neuroinflammatory condition classification based on the probability; and generate a predicted diagnosis for the patient based on the neuroinflammatory condition classification.

Example 2 is the computing apparatus of any previous or subsequent Example, wherein the one or more antigen receptor sequences associated with the patient comprise one of: (a) a B-cell receptor (BCR) immunoglobulin heavy chain (IGH) deoxyribonucleic acid (DNA) sequence; (b) an immunoglobulin light chain DNA sequence selected from the lambda (IGL) or kappa (IGK) chains; or (c) a T-cell receptor (TCR) DNA sequence selected from the alpha (TCRα) or beta (TCRβ) chains.

Example 3 is the computing apparatus of any previous or subsequent Example, wherein: the processor-executable instructions to identify the immune signature within the one or more antigen receptor sequences, when executed by the processor, further direct the computing apparatus to generate one or more probabilities that the one or more antigen receptor sequences correspond to clonality of B-cell receptors or T-cell receptors; and the processor-executable instructions to classify the one or more antigen receptor sequences into the neuroinflammatory condition classification based on the probability further direct the computing apparatus to classify the immune signature into the neuroinflammatory condition classification based on the one or more probabilities.

Example 4 is the computing apparatus of any previous or subsequent Example, wherein: the processor-executable instructions to receive the one or more antigen receptor sequences further direct the computing apparatus to receive a plurality of antigen receptor sequences from the patient; and the processor-executable instructions further direct the computing apparatus to generate a plurality of sequence embeddings from the plurality of antigen receptor sequences, wherein a sequence embedding of the plurality of sequence embeddings corresponds to a respective antigen receptor sequence of the plurality of antigen receptor sequences; determine a local density of the plurality of sequence embeddings; and generate, by the IS detection engine, a single vector representing an antigen receptor repertoire of the patient using the plurality of sequence embeddings and the local density.

Example 5 is the computing apparatus of any previous or subsequent Example, wherein the processor-executable instructions to identify the immune signature further direct the computing apparatus to process, by an autoregressive language model, the single vector representing the antigen receptor repertoire of the patient and identify the immune signature from an output of the autoregressive language model.

Example 6 is the computing apparatus of any previous or subsequent Example, wherein: the one or more antigen receptor sequences associated with the patient comprise a B-cell receptor (BCR) immunoglobulin heavy chain (IGH) deoxyribonucleic acid (DNA) sequence; and the processor-executable instructions further direct the computing apparatus to classify the one or more antigen receptor sequences into a multiple sclerosis (MS) classification.

Example 7 is a method comprising: receiving, by an immune signature (IS) detection engine, one or more antigen receptor sequences of a first patient; processing, using a language model of the IS detection engine, the one or more antigen receptor sequences; determining, by the IS detection engine, a first immune signature within the one or more antigen receptor sequences of the first patient; and detecting, by the IS detection engine, a first neuroinflammatory condition based on the first immune signature.

Example 8 is the method of any previous or subsequent Example, wherein: receiving the one or more antigen receptor sequences further comprises receiving a plurality of antigen receptor sequences from the first patient; processing the one or more antigen receptor sequences further comprises generating, by the language model, a plurality of sequence embeddings corresponding to the plurality of antigen receptor sequences; generating a single vector representing an antigen receptor repertoire of the first patient using the plurality of sequence embeddings; and determining the first immune signature comprises processing, by a second language model, the single vector to determine the first immune signature.

Example 9 is the method of any previous or subsequent Example, wherein the method further comprises training the language model of the IS detection engine, wherein training the language model comprises submitting a first training dataset into the language model; determining a plurality of misclassified antigen receptor sequences; identifying a subset of the plurality of misclassified antigen receptor sequences having a highest assigned probability; and training the language model by excluding the subset of misclassified antigen receptor sequences in a training loss calculation.

Example 10 is the method of any previous or subsequent Example, wherein processing the one or more antigen receptor sequences further comprises generating, by the language model, at least one clonality probability for the one or more antigen receptor sequences, wherein the at least one clonality probability indicates a likelihood that the one or more antigen receptor sequences correspond to B-cell clonality or T-cell clonality associated with the first neuroinflammatory condition.

Example 11 is the method of any previous or subsequent Example, wherein determining the first immune signature further comprises generating one or more probabilities that the one or more antigen receptor sequences correspond to clonality associated with the first neuroinflammatory condition, and the method further comprises classifying, by a classification model, the first immune signature into a first neuroinflammatory condition classification based on the one or more probabilities.

Example 12 is the method of any previous or subsequent Example, wherein the method further comprises receiving a probability that the one or more antigen receptor sequences comprise the first immune signature and classifying, using a logistic regression model of the IS detection engine, the probability into one of a plurality of neuroinflammatory condition classifications.

Example 13 is the method of any previous or subsequent Example, wherein the method further comprises receiving a second antigen receptor sequence of a second patient; processing the second antigen receptor sequence using the language model; determining a second immune signature within the second antigen receptor sequence; and classifying the second immune signature as corresponding to a healthy classification.

Example 14 is the method of any previous or subsequent Example, wherein the one or more antigen receptor sequences are acquired from a blood sample from the patient.

Example 15 is a non-transitory computer-readable storage medium comprising processor-executable instructions configured to cause a processor to: receive, by an immune signature (IS) detection engine, a plurality of antigen receptor sequences from a patient; generate, using a language model of the IS detection engine, a plurality of sequence embeddings from the plurality of antigen receptor sequences; determine, by the IS detection engine, an immune signature based on the plurality of sequence embeddings; and determine, by the IS detection engine, a neuroinflammatory condition of the patient corresponding to the immune signature.

Example 16 is the non-transitory computer-readable storage medium of any previous or subsequent Example, wherein the processor-executable instructions further cause the processor to generate a vector representation of the plurality of antigen receptor sequences, wherein the vector representation represents an antigen receptor repertoire of the patient; generate a probability that the vector representation corresponds to the neuroinflammatory condition; and classify the vector representation into a neuroinflammatory condition classification based on the probability.

Example 17 is the non-transitory computer-readable storage medium of any previous or subsequent Example, wherein the processor-executable instructions further cause the processor to generate a probability that a first antigen receptor sequence of the plurality of antigen receptor sequences corresponds to clonality, wherein the probability indicates a likelihood that the first antigen receptor sequence corresponds to B-cell clonality or T-cell clonality associated with the neuroinflammatory condition.

Example 18 is the non-transitory computer-readable storage medium of any previous or subsequent Example, wherein the processor-executable instructions further cause the processor to classify, via a logistic regression model, the probability that the first antigen receptor sequence corresponds to public clonality or private clonality into a neuroinflammatory condition classification corresponding to the neuroinflammatory condition.

Example 19 is the non-transitory computer-readable storage medium of any previous or subsequent Example, wherein the processor-executable instructions further cause the processor to receive a probability distribution corresponding to the immune signature; apply one of a logistic regression model or random forest model to the probability distribution; and classify the immune signature into one of a plurality of neuroinflammatory condition classifications based on an output from the logistic regression model or the random forest model.

Example 20 is the non-transitory computer-readable storage medium of any previous or subsequent Example, wherein the IS detection engine comprises an autoregressive language model, and the processor-executable instructions further cause the processor to train the autoregressive language model using a training dataset comprising: a first plurality of antigen receptor sequences corresponding to a plurality of neuroinflammatory conditions; a second plurality of antigen receptor sequences corresponding to healthy controls; a first set of labels corresponding to the neuroinflammatory conditions; and a second set of labels corresponding to a healthy classification for the healthy controls; wherein training the autoregressive language model comprises submitting a first batch of the training dataset as input to the autoregressive language model; determining a plurality of misclassified antigen receptor sequences in the first batch; ranking the plurality of misclassified antigen receptor sequences by predicted probability that the respective antigen receptor sequence corresponds to the healthy classification; selecting a subset of misclassified antigen receptor sequences having highest predicted probabilities; and excluding the subset of misclassified antigen receptor sequences from a loss calculation used to train the autoregressive language model.

What is claimed is:

1. A computing apparatus comprising:
a computer-readable storage medium;
an immune signature (IS) detection engine comprising processor-executable instructions stored on the computer-readable storage medium; and
a processor coupled to the computer-readable storage medium and configured to execute the processor-executable instructions, wherein the processor-executable instructions, when executed by the processor, direct the computing apparatus to at least:
receive one or more antigen receptor sequences associated with a patient;
identify an immune signature within the one or more antigen receptor sequences;
generate a probability that the immune signature corresponds to one of a plurality of neuroinflammatory conditions;
classify the one or more antigen receptor sequences into a neuroinflammatory condition classification based on the probability; and
generate a predicted diagnosis for the patient based on the neuroinflammatory condition classification.

2. The computing apparatus of claim 1, wherein the one or more antigen receptor sequences of the patient comprises one or more of:
(a) a B-cell receptor (BCR) immunoglobulin heavy chain (IGH) deoxyribonucleic acid (DNA) sequence;(b) an immunoglobulin light chain DNA sequence, selected from the lambda (IGL) or kappa (IGK) chains; or(c) a T-cell receptor (TCR) DNA sequence, selected from the alpha (TCRα) or beta (TCRβ) chains.

3. The computing apparatus of claim 1, wherein:
the processor-executable instructions to identify the immune signature within the one or more antigen receptor sequences, when executed by the processor, further direct the computing apparatus to:
generate one or more probabilities that the one or more antigen receptor sequences correspond to clonality of B-cell receptors or T-cell receptors; and
the processor-executable instructions to classify the one or more antigen receptor sequences into the neuroinflammatory condition classification based on the probability, when executed by the processor, further direct the computing apparatus to:
classify the immune signature into the neuroinflammatory condition classification based on the one or more probabilities.

4. The computing apparatus of claim 1, wherein:
the processor-executable instructions to receive the one or more antigen receptor sequences of the patient, when executed by the processor, further direct the computing apparatus to receive a plurality of antigen receptor sequences from the patient; and
the processor-executable instructions, when executed by the processor, further direct the computing apparatus to:
generate a plurality of sequence embeddings from the plurality of antigen receptor sequences, wherein a sequence embedding of the plurality of sequence embeddings corresponds to a respective antigen receptor sequence of the plurality of antigen receptor sequences;
determine a local density of the plurality of sequence embeddings; and
generate a single vector representing an antigen receptor repertoire of the patient using the plurality of sequence embeddings and the local density.

5. The computing apparatus of claim 1, wherein the processor-executable instructions to identify the immune signature within the one or more antigen receptor sequences, when executed by the processor, further direct the computing apparatus to:
process the one or more antigen receptor sequences using an autoregressive language model to generate a plurality of sequence embeddings; and
determine the immune signature for the patient based on the plurality of sequence embeddings.

6. The computing apparatus of claim 1, wherein:
the one or more antigen receptor sequences of the patient comprises a B-cell receptor (BCR) immunoglobulin heavy chain (IGH) deoxyribonucleic acid (DNA) sequence; and
the processor-executable instructions to classify the one or more antigen receptor sequences into a neuroinflammatory condition classification based on the probability, when executed by the processor, further direct the computing apparatus to:
classify the one or more antigen receptor sequences into a multiple sclerosis (MS) classification.

7. A method comprising:
receiving, by an immune signature (IS) detection engine, one or more antigen receptor sequences of a first patient;
processing, using a language model of the IS detection engine, the one or more antigen receptor sequences;
determining, by the IS detection engine, a first immune signature within the one or more antigen receptor sequences of the first patient; and
detecting, by the IS detection engine, a first neuroinflammatory condition based on the first immune signature.

8. The method of claim 7, wherein:
receiving, by the IS detection engine, the one or more antigen receptor sequences of the first patient further comprises:
receiving a plurality of antigen receptor sequences from the first patient;
processing, using the language model of the IS detection engine, the one or more antigen receptor sequences further comprises:
generating, by the language model, a plurality of sequence embeddings of the plurality of antigen receptor sequences, wherein a sequence embedding of the plurality of sequence embeddings corresponds to a respective antigen receptor sequence of the plurality of antigen receptor sequences; and
generating a single vector representing an antigen receptor repertoire of the first patient using the plurality of sequence embeddings; and
determining, by the IS detection engine, the first immune signature within the one or more antigen receptor sequences comprises:
processing, by a second language model of the IS detection engine, the single vector to determine the first immune signature within the plurality of antigen receptor sequences.

9. The method of claim 7, wherein the method further comprises training the language model of the IS detection engine, wherein training the language model of the IS detection engine comprises:
submitting a first training dataset into the language model;
determining a plurality of misclassified antigen receptor sequences;
identifying a subset of the plurality of misclassified antigen receptor sequences having a highest assigned probability; and
training the language model by excluding the subset of misclassified antigen receptor sequences from a training loss calculation.

10. The method of claim 7, wherein processing, via the language model of the IS detection engine, the one or more antigen receptor sequences further comprises:
generating, by the language model, at least one clonality probability for the one or more antigen receptor sequences, wherein the at least one clonality probability indicates a likelihood that the one or more antigen receptor sequences comprises B-cell clonality or T-cell clonality associated with the first neuroinflammatory condition.

11. The method of claim 7, wherein:
determining, by the IS detection engine, the first immune signature within the one or more antigen receptor sequences comprises:
generating, by the language model, one or more probabilities that the one or more antigen receptor sequences correspond to clonality associated to the first neuroinflammatory condition; and
the method further comprises:
classifying, by a classification model, the first immune signature into a first neuroinflammatory condition classification based on the one or more probabilities.

12. The method of claim 7, wherein the method further comprises:
receiving, from the language model of the IS detection engine, a probability that the one or more antigen receptor sequences comprises the first immune signature; and
classifying, using a logistic regression model of the IS detection engine, the probability into a neuroinflammatory condition classification.

13. The method of claim 7, wherein the method further comprises:
receiving, by the IS detection engine, a second antigen receptor sequence of a second patient;
processing, via the language model of the IS detection engine, the second antigen receptor sequence;
determining, by the IS detection engine, a second immune signature within the second antigen receptor sequence of the second patient; and
classifying, by the IS detection engine, the second immune signature as corresponding to a healthy classification.

14. The method of claim 7, wherein the one or more antigen receptor sequences are acquired from a blood sample from the patient.

15. A non-transitory computer-readable storage medium comprising processor-executable instructions configured to cause a processor to:
receive, by an immune signature (IS) detection engine, a plurality of antigen receptor sequences from a patient;
generate, using a language model of the IS detection engine, a plurality of sequence embeddings from the plurality of antigen receptor sequences;
determine, by the IS detection engine, an immune signature based on the plurality of sequence embeddings; and
determine, by the IS detection engine, a neuroinflammatory condition of the patient corresponding to the immune signature.

16. The non-transitory computer-readable storage medium of claim 15, wherein the processor-executable instructions further cause the processor to:
generate a vector representation of the plurality of antigen receptor sequences, wherein the vector representation represents an antigen receptor repertoire of the patient;
generate a probability that the vector representation corresponds to the neuroinflammatory condition; and
classify the vector representation into a neuroinflammatory condition classification based on the probability.

17. The non-transitory computer-readable storage medium of claim 15, wherein the processor-executable instructions to generate, by the IS detection engine, the immune signature from the plurality of antigen receptor sequences further cause the processor to:
generate, by the IS detection engine, a probability that a first antigen receptor sequence of the plurality of antigen receptor sequences corresponds to clonality, wherein:
the probability that the first antigen receptor sequence corresponds to the clonality indicates a likelihood that the first antigen receptor sequence corresponds to B-cell clonality or T-cell clonality associated with the neuroinflammatory condition.

18. The non-transitory computer-readable storage medium of claim 17, wherein the processor-executable instructions cause the processor to further execute processor-executable instructions stored in the non-transitory computer-readable storage medium to:
classify, via a logistic regression model, the probability that the first antigen receptor sequence corresponds to public clonality or private clonality into a first neuroinflammatory condition classification corresponding to the neuroinflammatory condition.

19. The non-transitory computer-readable storage medium of claim 15, wherein the processor-executable instructions to determine, by the IS detection engine, the neuroinflammatory condition corresponding to the immune signature cause the processor to further execute processor-executable instructions stored in the non-transitory computer-readable storage medium to:
receive a probability distribution corresponding to the immune signature;
apply one of a logistic regression model or random forest model to the probability distribution; and
classify the immune signature into one of a plurality of neuroinflammatory condition classifications based on an output from the logistic regression model or the random forest model.

20. The non-transitory computer-readable storage medium of claim 15, wherein the IS detection engine comprises an autoregressive language model and the processor-executable instructions further cause the processor to:
train the autoregressive language model using a training dataset comprising:
a first plurality of antigen receptor sequences corresponding to a plurality of neuroinflammatory conditions;
a second plurality of antigen receptor sequences corresponding to healthy controls;
a first set of labels corresponding to the one or more neuroinflammatory conditions; and
a second set of labels corresponding to a healthy classification for the healthy controls; and
wherein training the autoregressive language model comprises:
submitting a first batch of the training dataset as an input to the autoregressive language model;
determining a plurality of misclassified antigen receptor sequences in the first batch, wherein the plurality of misclassified antigen receptor sequences comprises a subset of the first plurality of antigen receptor sequences classified into the healthy classification;
ranking the plurality of misclassified antigen receptor sequences by predicted probability that the respective antigen receptor sequence corresponds to the healthy classification;
selecting a subset of misclassified antigen receptor sequences having a highest predicted probability; and
excluding the subset of misclassified antigen receptor sequences from a loss calculation used to train the autoregressive language model.
